(19) United States
(12) Patent Application Publication
(10) Pub. No.: US 2026/0096769 A1
(43) Pub. Date: Apr. 9, 2026

(54) SYSTEMS AND METHODS FOR EVALUATING STATE OF CARDIAC MONITORING DEVICES

(71) Applicant: Medtronic, Inc., Minneapolis, MN (US)

(72) Inventors: Veronica Ramos, Miami, FL (US); Sean R. Landman, Minneapolis, MN (US); Grant A. Neitzell, Loretto, MN (US); Kyle R. Zablocki, Jamesburg, NJ (US); Gerard L. Torenvliet, Hugo, MN (US); Chris S. Kulseth, Farmington, MN (US); April M. Walters, North Oaks, MN (US); Dillon A. Holm, Hudson, WI (US); Kaja S. Pederson, Farmington, MN (US); Jennifer N. Koomen, Stillwater, MN (US); Maria F. Cruz, Minneapolis, MN (US)

(21) Appl. No.: 19/341,186

(22) Filed: Sep. 26, 2025

Related U.S. Application Data
(60) Provisional application No. 63/704,407, filed on Oct. 7, 2024.

(51) Int. Cl. A61B 5/363; A61B 5/00; G16H 50/30

(52) U.S. Cl. CPC A61B 5/363; A61B 5/6869; A61B 5/7221; G16H 50/30

(57) ABSTRACT

Various embodiments relate to a method and related device and computer-readable storage medium for evaluating a cardiac monitoring device including one or more of the following: receiving an EGM signal from a cardiac monitoring device inserted in a patient, predicting a plurality of potential cardiac episodes experienced by the patient based on the EGM signal, and analyzing the plurality of potential cardiac episodes to evaluate a state of the cardiac monitoring device. For example, the method may include characterizing a health state (e.g., device performance) and/or determining whether a change in device settings may be warranted, based on the predicted cardiac episodes.

### RELATED APPLICATIONS

This application claims the benefit of U.S. Provisional Application Ser. No. 63/704,407, filed Oct. 7, 2024, the entire contents of each of which are incorporated herein by reference.

### TECHNICAL FIELD

The present technology relates to systems and methods for evaluating state of cardiac monitoring devices.

### BACKGROUND

Cardiac monitoring devices may be used to detect and track frequency and type of any cardiac episodes experienced in a patient suspected of having cardiac issues such as cardiac rhythm issues (e.g., arrythmia, atrial fibrillation, etc.). Cardiac monitoring of patients both inside and outside of clinical settings has become more prevalent, with cardiac monitoring devices being utilized to detect arrhythmic conditions (e.g., abnormality or perturbation in the normal electrical rhythm of the heart). For example, an insertable cardiac monitor (ICM) may be implanted in a patient and include one or more sensors configured to collect physiological data, such as electrocardiogram (ECG) and/or electrogram (EGM) sensors for detecting cardiac activity. Clinicians may utilize this physiological data to characterize the cardiac activity of the patient, such as for diagnosing or evaluating the progression of cardiac issues.

However, an ICM may experience changes in signal quality over time, due to reasons such as hardware degradation or failure that result in a decline in device performance. Because ICM-recorded signals often include some amount of noise due to the implant location of the ICM and/or other factors, clinicians may not realize when noise becomes significant and/or sustained enough to possibly indicate a serious device performance issue.

### SUMMARY

The subject technology is illustrated, for example, according to various aspects described below, including with reference to FIGS. 1-11. Various examples of aspects of the subject technology are described as numbered clauses (1, 2, 3, etc.) for convenience. These are provided as examples and do not limit the subject technology.

Various embodiments described herein related to method including one or more of the following: receiving an electrogram (EGM) signal from a cardiac monitoring device inserted in a patient; predicting a plurality of potential cardiac episodes experienced by the patient, based on the EGM signal; and characterizing a health state of the cardiac monitoring device, based at least in part on the plurality of predicted potential cardiac episodes.

Various embodiments are described wherein characterizing a health state of the cardiac monitoring device comprises assessing quality of the EGM signal.

Various embodiments are described wherein characterizing a health state of the cardiac monitoring device comprises determining a performance degradation of the cardiac monitoring device.

Various embodiments are described wherein predicting a plurality of potential cardiac episodes comprises identifying one or more potential arrhythmia episodes from the EGM signal.

Various embodiments are described wherein the one or more arrhythmia episodes comprises at least one of ventricular tachyarrhythmia, bradyarrhythmia, asystole, atrial fibrillation, or atrial tachyarrhythmia.

Various embodiments are described wherein characterizing a health state of the cardiac monitoring device comprises evaluating a set of one or more device metrics associated with the plurality of predicted potential cardiac episodes, with a portion of the EGM signal received over a period of interest, or both.

Various embodiments are described wherein characterizing a health state of the cardiac monitoring device comprises determining whether the cardiac monitoring device is providing an EGM signal having at least a threshold amount of noise, wherein the threshold amount of noise is associated with at least one of the device metrics.

Various embodiments are described wherein the threshold amount of noise is associated with at least one of: number of potential fast-type ventricular tachyarrhythmia episodes that are confirmed as a fast-type ventricular tachyarrhythmia episode (confirmed FVT episode); or number of potential fast-type ventricular tachyarrhythmia episodes that are reclassified as a noise event (rejected FVT episode).

Various embodiments are described wherein characterizing a health state of the cardiac monitoring device further comprises, in response to determining that the cardiac monitoring device is providing an EGM signal having at least a threshold amount of noise: evaluating a pattern of the set of device metrics; and flagging the cardiac monitoring device as faulty if the pattern of the set of device metrics includes a simultaneous change in a plurality of device metrics.

Various embodiments are described wherein evaluating a pattern of the set of device metrics comprises evaluating one or more of: number of confirmed FVT episodes per day, number of rejected FVT episodes per day, duration of confirmed FVT episodes per day, or noise level in the portion of the EGM signal received over the period of interest.

Various embodiments are described wherein characterizing a health state of the cardiac monitoring device comprises flagging the cardiac monitoring device as faulty if the simultaneous change in the plurality of device metrics is sustained for at least a threshold period of time.

Various embodiments are described wherein characterizing a health state of the cardiac monitoring device comprises flagging the cardiac monitoring device as faulty if an occurrence rate of predicted cardiac episodes exceeds a predetermined threshold.

Various embodiments are described further comprising recommending an action based on the health state of the cardiac monitoring device.

Various embodiments are described wherein the recommended action comprises removal of the cardiac monitoring device from the patient.

Various embodiments are described wherein the recommended action comprises issuing a recall of distributed cardiac monitoring devices.

Various embodiments described herein relate to a system including one or more of the following: a processor; a memory operably coupled to the processor and storing instructions that, when executed by the processor, cause the system to perform operations comprising: receiving an electrogram (EGM) signal from a cardiac monitoring device inserted in a patient; predicting a plurality of potential cardiac episodes experienced by the patient, based on the EGM signal; and characterizing a health state of the cardiac monitoring device, based at least in part on the plurality of predicted potential cardiac episodes.

Various embodiments are described wherein characterizing a health state of the cardiac monitoring device comprises assessing quality of the EGM signal.

Various embodiments are described wherein characterizing a health state of the cardiac monitoring device comprises determining a performance degradation of the cardiac monitoring device.

Various embodiments are described wherein characterizing a health state of the cardiac monitoring device comprises evaluating a set of one or more device metrics associated with the plurality of predicted potential cardiac episodes, a portion of the EGM signal received over a period of interest, or both.

Various embodiments are described wherein characterizing a health state of the cardiac monitoring device comprises determining whether the cardiac monitoring device is providing an EGM signal having at least a threshold amount of noise, wherein the threshold amount of noise is associated with a set of one or more device metrics.

Various embodiments are described wherein the threshold amount of noise is associated with at least one of: number of potential fast-type ventricular tachyarrhythmia episodes that are confirmed as a fast-type ventricular tachyarrhythmia episode (confirmed FVT episode); or number of potential fast-type ventricular tachyarrhythmia episodes that are reclassified as a noise event (rejected FVT episode).

Various embodiments are described wherein characterizing a health state of the cardiac monitoring device further comprises, in response to determining that the cardiac monitoring device is providing an EGM signal having at least a threshold amount of noise: evaluating a pattern of the set of device metrics; and flagging the cardiac monitoring device as faulty if the pattern of the set of device metrics includes a simultaneous change in a plurality of device metrics.

Various embodiments are described wherein characterizing a health state of the cardiac monitoring device comprises flagging the cardiac monitoring device as faulty if the simultaneous change in the plurality of device metrics is sustained for at least a threshold period of time.

Various embodiments are described wherein characterizing a health state of the cardiac monitoring device comprises flagging the cardiac monitoring device as faulty if a detection rate of predicted cardiac episodes exceeds a predetermined threshold.

Various embodiments are described wherein the cardiac monitoring device comprises a subcutaneous cardiac monitoring device.

Various embodiments described herein relate to a method including one or more of the following: receiving an electrogram (EGM) signal from a cardiac monitoring device inserted in a patient; predicting a plurality of potential cardiac episodes experienced by the patient, based on the EGM signal; and recommending a change in one or more settings of the cardiac monitoring device, based at least in part on the plurality of predicted potential cardiac episodes.

Various embodiments are described wherein predicting a plurality of potential cardiac episodes comprises identifying one or more potential arrhythmia episodes from the EGM signal.

Various embodiments are described wherein the one or more arrhythmia episodes comprises at least one of ventricular tachyarrhythmia, bradyarrhythmia, asystole, atrial fibrillation, or atrial tachyarrhythmia.

Various embodiments are described wherein recommending a change in one or more settings of the cardiac monitoring device is based at least in part on a detection rate of the predicted potential cardiac episodes.

Various embodiments are described wherein the recommended change comprises activating alerts for detection of a first cardiac episode type, in response to a detection rate of cardiac episodes of the first cardiac episode type being below a first predetermined threshold.

Various embodiments are described wherein the first cardiac episode type is atrial fibrillation and the detection rate of cardiac episodes of the first cardiac episode type is evaluated after an atrial fibrillation treatment.

Various embodiments are described wherein the recommended change comprises deactivating alerts for detection of a second cardiac episode type, in response to a detection rate of cardiac episodes of the second cardiac episode type exceeding a second predetermined threshold.

Various embodiments are described wherein the recommended change comprises, in response to a detection rate of cardiac episodes of a third cardiac episode type exceeding a third predetermined threshold, increasing the specificity for detecting cardiac episodes of the third cardiac episode type.

Various embodiments are described wherein the recommended change comprises, in response to a detection rate of cardiac episodes of a fourth cardiac episode type exceeding a fourth predetermined threshold, allocating more device memory for storing data associated with the fourth cardiac episode type.

Various embodiments are described wherein the recommended change comprises modifying a cardiac episode detection profile, in response to the predicted potential cardiac episodes.

Various embodiments are described wherein modifying the cardiac episode detection profile comprises activating alerts for one or more cardiac episode types, deactivating alerts for one or more cardiac episode types, or modifying a threshold associated with detection of one or more cardiac episode types.

Various embodiments are described wherein the recommended change is further based on a portion of the EGM signal received over a period of interest.

Various embodiments described herein relate to a cardiac monitoring system, including one or more of the following: a processor; a memory operably coupled to the processor and storing instructions that, when executed by the processor, cause the system to perform operations comprising: receiving an electrogram (EGM) signal from a cardiac monitoring device inserted in a patient; predicting a plurality of potential cardiac episodes experienced by the patient, based on the EGM signal; and recommending a change in one or more settings of the cardiac monitoring device, based at least in part on the plurality of predicted potential cardiac episodes.

Various embodiments are described wherein predicting a plurality of potential cardiac episodes comprises identifying one or more potential arrhythmia episodes from the EGM signal.

Various embodiments are described wherein recommending a change in one or more settings of the cardiac monitoring device is based at least in part on a detection rate of the predicted potential cardiac episodes.

Various embodiments are described wherein the recommended change comprises activating alerts for detection of a first cardiac episode type, in response to a detection rate of cardiac episodes of the first cardiac episode type being below a first predetermined threshold.

Various embodiments are described wherein the first cardiac episode type is atrial fibrillation and the detection rate of cardiac episodes of the first cardiac episode type is evaluated after an atrial fibrillation treatment.

Various embodiments are described wherein the recommended change comprises deactivating alerts for detection of a second cardiac episode type, in response to a detection rate of cardiac episodes of the second cardiac episode type exceeding a second predetermined threshold.

Various embodiments are described wherein the recommended change comprises, in response to a detection rate of cardiac episodes of a third cardiac episode type exceeding a third predetermined threshold, increasing the third predetermined threshold.

Various embodiments are described wherein the recommended change comprises, in response to a detection rate of cardiac episodes of a fourth cardiac episode type exceeding a fourth predetermined threshold, allocating more device memory for storing data associated with the fourth cardiac episode type.

Various embodiments are described wherein the recommended change comprises modifying a cardiac episode detection profile, in response to the predicted potential cardiac episodes.

Various embodiments are described wherein modifying the cardiac episode detection profile comprises activating alerts for one or more cardiac episode types, deactivating alerts for one or more cardiac episode types, or modifying a threshold associated with detection of one or more cardiac episode types.

Various embodiments are described wherein the recommended change is further based on a portion of the EGM signal received over a period of interest.

Various embodiments are described wherein the cardiac monitoring device comprises a subcutaneous cardiac monitoring device.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1 is a schematic flowchart of an example variation of a method for evaluating a state of a cardiac monitoring device, in accordance with the present technology.

FIG. 2 is a schematic flowchart of an example variation of a method for evaluating a state of a cardiac monitoring device, in accordance with the present technology.

FIG. 3 is an illustrative schematic of an example variation of an insertable cardiac monitoring device, in accordance with the present technology.

FIG. 4 is a conceptual diagram of an example variation of an insertable cardiac monitoring device, in accordance with the present technology.

FIG. 5 is an illustrative schematic of a cardiac monitoring system including an insertable cardiac monitoring device placed in a patient, in accordance with the present technology.

FIG. 6 is a schematic flowchart of an example process of characterizing a health state of a cardiac monitoring device based on cardiac episode-related metrics, in accordance with the present technology.

FIGS. 7-11 are example plots of device metrics derived from longitudinal EGM data for respective cardiac monitoring devices, for use in evaluating state of the respective cardiac monitoring devices.

### DETAILED DESCRIPTION

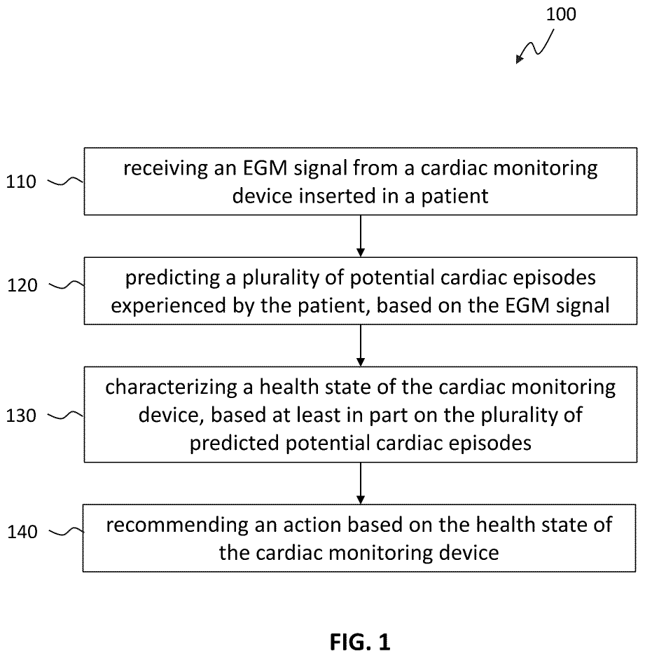

The present technology relates to systems and methods for evaluating state of cardiac monitoring device. Some variations of the present technology, for example, are directed to evaluating health state, suitable device settings for cardiac episode detection, and/or the like. Specific details of several variations of the technology are described below with reference to FIGS. 1-11.

Cardiac monitoring devices are used to measure electrical activity of the heart and record cardiac information of a patient such as heart rate and rhythm. The cardiac electrical activity data is primarily referred to herein as electrogram (EGM) data, although for the methods and systems described herein the cardiac electrical activity may additionally or alternatively include electrocardiogram (ECG) or other signal data. The EGM data can be analyzed, for example, to detect the occurrence of cardiac events experienced by the patient, and may be used by clinicians to diagnose cardiac disorders (e.g., cardiac rhythm disorders). Some cardiac monitoring devices are wearable (e.g., patch-based and applied to a skin surface of the chest, otherwise applied to the patient on an as-needed basis such as when cardiac symptoms arise), while some cardiac monitoring devices are inserted into the patient for longer term monitoring such as up to several years of cardiac monitoring.

Insertable cardiac monitoring devices, as well as other kinds of cardiac monitoring devices, can degrade over time due to issues such as hardware failure, which can cause the cardiac monitoring device to experience undesirable changes in signal quality (e.g., excessive noise) and lead to poor monitoring device performance. For example, various factors can affect the fidelity of the EGM signal of an insertable cardiac monitoring device. A low-quality signal might be translated into lower Rwaves, making the device prone to over- or under-sensing cardiac episodes experienced by the patient and/or false detections of cardiac episodes experienced by the patient.

Because recorded EGM signals inherently often include some amount of noise due to common factors such as suboptimal implant location of the insertable cardiac monitoring device, patient movement, and/or environmental noise, a clinician is typically used to observing noise in these signals and thus may not realize when noise becomes more significant and/or sustained and ultimately affect device performance. Due to the implanted nature of the insertable cardiac monitoring device, it may be challenging to immediately identify any hardware issues that may be contributing to poor device performance. As such, it may be desirable to assess device state of an insertable cardiac monitoring device placed in a patient. Furthermore, it may be desirable to modify device detection settings based on changing circumstances surrounding the operation of the cardiac monitoring device, such as to better suit monitoring objectives for the patient.

Generally, as described in further detail herein, a state of the cardiac monitoring device may be characterized at least in part on the EGM signal provided by the cardiac monitoring device, and in some variations based at least in part on metrics associated with cardiac episodes that are predicted or otherwise determined based on such EGM signal.

For example, FIG. 1 is a schematic flowchart an example method 100 for evaluating a state of a cardiac monitoring device. As shown in FIG. 1, the method 100 may include receiving an EGM signal from a cardiac monitoring device 110, predicting a plurality of potential cardiac episodes experienced by the patient based on the EGM signal 120, and characterizing a health state of the cardiac monitoring device 130, based at least in part on the plurality of predicted potential cardiac episodes. As described in further detail herein, the characterization of the device's health state may, for example, be related to quality of the EGM signal that is provided by the cardiac monitoring device and/or other performance level (e.g., degradation) of the cardiac monitoring device (e.g., caused by hardware and/or software issues in the cardiac monitoring device). Based on the characterization of the health state, the cardiac monitoring device may be flagged as faulty, and may trigger a recommended action such as removal and/or replacement of the cardiac monitoring device.

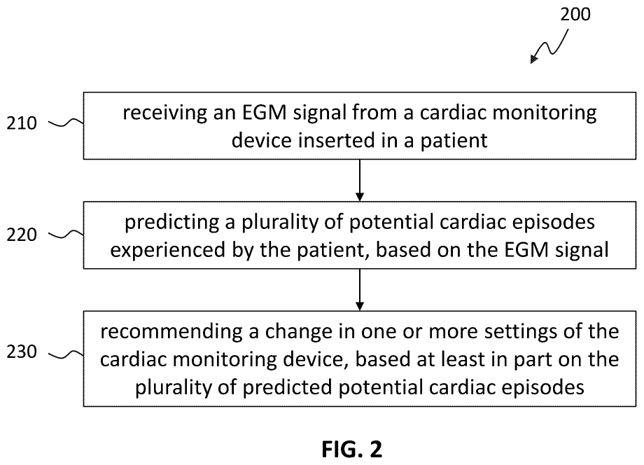

FIG. 2 is a schematic flowchart of another example method 200 for evaluating a state of a cardiac monitoring device. As shown in FIG. 2, the method 200 may include receiving an EGM signal from a cardiac monitoring device 210, predicting a plurality of potential cardiac episodes experienced by the patient based on the EGM signal 220, and recommending a change in one or more settings of the cardiac monitoring device 230, based at least in part on the plurality of predicted potential cardiac episodes.

Further aspects of example cardiac monitoring systems and devices, and methods for evaluating the state thereof, are described in further detail below.

### I. Cardiac Monitoring Devices and Systems

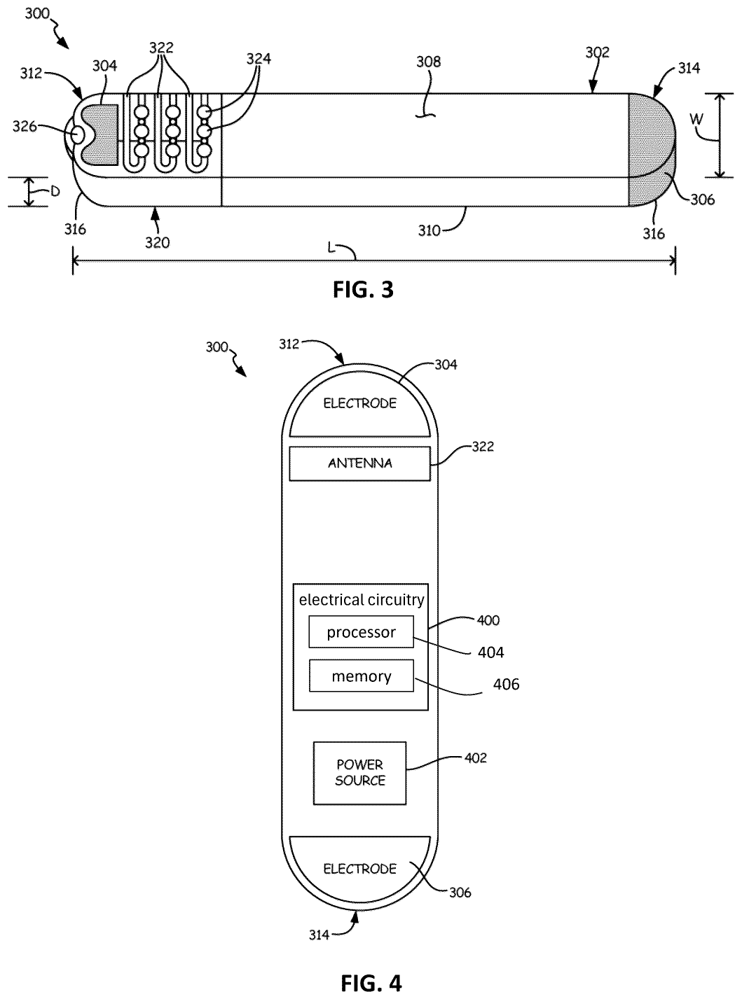

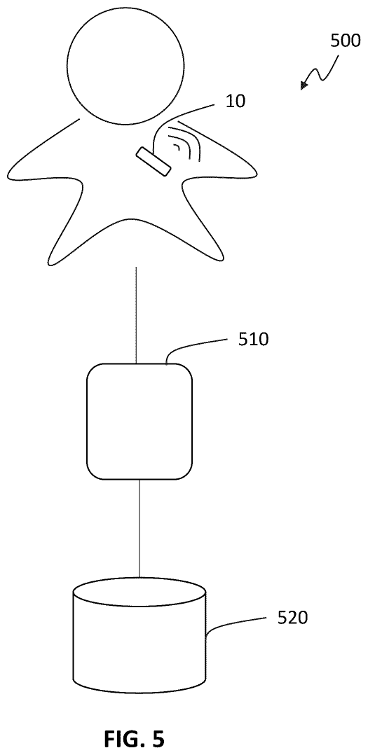

FIG. 3 is a conceptual diagram of an example of an insertable cardiac monitor (ICM) 300 (also referred to herein as a “cardiac monitoring device”) for detecting a bradycardia/asystole event, according to another variation of the present disclosure. In the example shown in FIG. 3, insertable cardiac monitor 300 may be embodied as a monitoring device having housing 302, a first (e.g., proximal) electrode 304, and a second (e.g., distal) electrode 306. Housing 302 may further comprise a first major surface 308, a second major surface 310, a first (e.g., proximal) end 312, and a second (e.g., distal) end 314. Housing 302 encloses electronic circuitry 400 and power source 402 (shown in FIG. 4) located inside the insertable cardiac monitor 300 and protects the circuitry contained therein from body fluids. Electrical feedthroughs provide electrical connection of electrodes 304 and 306.

In some variations such as that shown in FIG. 3, insertable cardiac monitor 300 is defined by a length L, a width W and thickness or depth D. The ICM may be in the form of an elongated rectangular prism wherein the length L is much larger than the width W, which in turn is larger than the depth D. In some variations, the geometry of the insertable cardiac monitor 300 (for example, a width W greater than the depth D) may be selected to allow the cardiac monitor 300 to be inserted under the skin of the patient using a minimally invasive procedure and to remain in the desired orientation during insert. For example, the device shown in FIG. 3 may include radial asymmetries (notably, the rectangular shape) along the longitudinal axis that maintains the device in the proper orientation following insertion. For example, in some variations the spacing between the proximal electrode 304 and distal electrode 306 may range from 30 millimeters (mm) to 55 mm, 35 mm to 55 mm, and from 40 mm to 55 mm and may be any range or individual spacing from 25 mm to 60 mm. In addition, insertable cardiac monitor 300 may have a length L that ranges from 30 mm to about 70 mm. In other variations, the length L may range from 40 mm to 60 mm, 45 mm to 60 mm and may be any length or range of lengths between about 30 mm and about 70 mm. In addition, the width W of major surface 308 may range from 3 mm to 10 mm and may be any single or range of widths between 3 mm and 10 mm. In some variations, the thickness of depth D of the insertable cardiac monitor 300 may range from 2 mm to 9 mm. For example, the depth D of the insertable cardiac monitor 300 may range from 2 mm to 5 mm and may be any single or range of depths from 2 mm to 9 mm. In addition, insertable cardiac monitor 300 according to an example variation of the present invention has a geometry and size designed for ease of implant and patient comfort. Variations of insertable cardiac monitor 300 described in this disclosure may have a volume of three cubic centimeters (cm) or less, 1.5 cubic cm or less or any volume between three and 1.5 cubic centimeters.

In the example shown in FIG. 3, once inserted within the patient, the first major surface 308 faces outward, toward the skin of the patient while the second major surface 310 is located opposite the first major surface 308. In addition, in the example shown in FIG. 3, proximal end 312 and distal end 314 are rounded to reduce discomfort and irritation to surrounding tissue once inserted under the skin of the patient. Insertable cardiac monitor 300, including instrument and method for inserting monitor 300 is described, for example, in U.S. Patent Publication No. 2014/0276928, incorporated herein by reference in its entirety.

As described with other variations, proximal electrode 304 and distal electrode 306 may be used to sense cardiac signals for determining a cardiac event (e.g., bradycardia or asystole event) such as EGM signals, intra-thoracically or extra-thoracically, which may be sub-muscularly or subcutaneously. EGM signals may be stored in a memory of the insertable cardiac monitor 300, and EGM data may be transmitted via integrated antenna 322 to another medical device, which may be another implantable device or an external device.

In the example variation shown in FIG. 3, proximal electrode 304 is in close proximity to the proximal end 312 and distal electrode 306 is in close proximity to distal end 314. In this variation, distal electrode 306 is not limited to a flattened, outward facing surface, but may extend from first major surface 308 around rounded edges 316 and onto the second major surface 310 so that the electrode 306 has a three-dimensional curved configuration. In the example variation shown in FIG. 3, proximal electrode 304 is located on first major surface 308 and is substantially flat, outward facing. However, in other variations, proximal electrode 304 may utilize the three-dimensional curved configuration similar to that of distal electrode 306, providing a three-dimensional proximal electrode (not shown in this variation). Additionally or alternatively, in other variations, distal electrode 306 may utilize a substantially flat, outward facing electrode located on first major surface 308 similar to that shown with respect to proximal electrode 304. The various electrode configurations allow for configurations in which proximal electrode 304 and distal electrode 306 are located on both first major surface 308 and second major surface 310. In other configurations, such as that shown in FIG. 3, only one of proximal electrode 304 and distal electrode 306 is located on both major surfaces 308 and 310, and in still other configurations both proximal electrode 304 and distal electrode 306 are located on one of the first major surface 308 or the second major surface 310 (e.g., proximal electrode 304 located on first major surface 308 while distal electrode 306 is located on second major surface 310). In some variations, the insertable cardiac monitor 300 may include electrodes on both major surface 308 and 310 at or near the proximal and distal ends of the device, such that a total of at least four electrodes are included on cardiac monitor device 300. Electrodes 304 and 306 may be formed of a plurality of different types of biocompatible conductive material (e.g. stainless steel, titanium, platinum, iridium, or alloys thereof), and/or may utilize one or more coatings such as titanium nitride or fractal titanium nitride.

In the example shown in FIG. 3, proximal end 312 includes a header assembly 320 that includes one or more of proximal electrode 304, an integrated antenna 322, anti-migration projections 324, and/or suture hole 326. The integrated antenna 322 may be located on the same major surface (e.g., first major surface 308) as proximal electrode 304 and may also be included as part of header assembly 320. Integrated antenna 322 allows insertable cardiac monitor 300 to transmit and/or receive data. In some variations, integrated antenna 322 may be formed on the opposite major surface as proximal electrode 304, or may be incorporated within the housing 322 of insertable cardiac monitor 300. In the example variation shown in FIG. 3, anti-migration projections 324 are located adjacent to integrated antenna 322 and protrude away from first major surface 308 to prevent longitudinal movement of the device, though may be arranged on any suitable surface of the insertable cardiac monitor 300. In the example variation shown in FIG. 3, anti-migration projections 324 include a plurality (e.g., nine) small bumps or protrusions extending away from first major surface 308; however, anti-migration projections 324 may additionally or alternatively be located on the opposite major surface as proximal electrode 304 and/or integrated antenna 322. As shown in FIG. 3, the suture hole 326, which may be used to help secure the insertable cardiac monitor 300 in the patient to prevent movement following insertion of the insertable cardiac monitor 300, may be located adjacent to proximal electrode 304, though one or more suture holes 326 may additionally or alternatively be located in any other suitable location. In some variations, the header assembly 320 is a molded header assembly made from a polymeric or plastic material, which may be integrated or separable from the main portion of insertable cardiac monitor 300.

FIG. 4 is a functional schematic diagram of the insertable cardiac monitor 300 as shown in FIG. 3 in accordance with the present technology. Insertable cardiac monitor 300 includes housing 302, proximal electrode 304 located at proximal end 312, distal electrode 306 located at distal end 314, integrated antenna 322, electrical circuitry 400 and power source 402. Electrical circuitry 400 may be coupled to proximal electrode 304 and distal electrode 306 to sense cardiac signals and monitor events (e.g., arrythmia, etc.). Electrical circuitry 400 is also connected to transmit and receive communications via integrated antenna 322. Power source 402 provides power to electrical circuitry 400, as well as to any other components that require power. Power source 402 may include one or more energy storage devices, such as one or more rechargeable or non-rechargeable batteries. The insertable cardiac monitor 300 as shown in FIGS. 3 and 4 may be a monitoring-only device. However, in other examples, insertable cardiac monitor 300 may further provide therapy delivery capabilities.

The electrical circuitry 400 receives raw EGM signals monitored by proximal electrode 304 and distal electrode 306. Electrical circuitry 400 may also include components/modules for converting the raw EGM signal to a processed EGM signal that can be analyzed to detect sense events. Although not shown, electrical circuitry 400 may include any discrete and/or integrated electronic circuit components that implement analog and/or digital circuits capable of producing the functions described for analyzing EGM signals to detect/verify bradycardia and/or asystole events. For example, the electrical circuitry 400 may include analog circuits, e.g., pre-amplification circuits, filtering circuits, and/or other analog signal conditioning circuits. The modules may also include digital circuits, e.g., digital filters, combinational or sequential logic circuits, state machines, integrated circuits, one or more processors 404 (shared, dedicated, or group) that executes one or more software or firmware programs, memory devices 406, or any other suitable components or combination thereof that provide the described functionality.

In some variations, electrical circuitry 400 may include a sensing unit for monitoring the EGM signal detected by the respective proximal and distal electrodes 304 and 306, respectively, and at least one sensing channel that utilizes an algorithm for identifying events in the EGM signal. For example, sensed events (e.g., R-waves) are utilized to detect one or more cardiac episodes. In some variations, electrical circuitry 400 includes a processor is utilized to receive information regarding the sensed events and implements one or more algorithms for determining whether a particular one or more events have occurred. In addition, the analog voltage signals received from electrodes 304 and 306 may be passed to analog-to-digital (A/D) converters included in the electrical circuitry 400, and stored in a memory unit 406 included as part of electrical circuitry 400 for subsequent analysis with firmware executed by the processor(s) 404 included as part of electrical circuitry 400.

Electrical circuitry 400 may control insertable cardiac monitor 300 functions and process EGM signals received from electrodes 304 and 306 according to programmed signal analysis routines or algorithms. The insertable cardiac monitor 300 may include other optional sensors (not shown) for monitoring physiological signals, such as an activity sensor, pressure sensor, oxygen sensor, accelerometer, and/or other sensor used to monitor a patient. These may also be provided to electrical circuitry 400 for processing.

Electrical circuitry 400 may similarly control monitoring time intervals and sampling rates according to a particular clinical application. In addition, electrical circuitry may include state machines or other sequential logic circuitry to control device functions and need not be implemented exclusively as a microprocessor. For example, electrical circuitry 400 may include timers utilized to detect asystole events as described in more detail below.

Electrical circuitry 400 communicates with integrated antenna 322 (shown in FIG. 3) or other communication to transmit electrical signal data, e.g. EGM signal data, stored in memory or received from electrical circuitry 400 in real time. Antenna 322 may be configured to transmit and receive communication signals via inductive coupling, electromagnetic coupling, tissue conductance, Near Field Communication (NFC), Radio Frequency Identification (RFID), BLUETOOTH®, WiFi, or other proprietary or non-proprietary wireless telemetry communication schemes.

The electrical circuitry 400 may include a communication module including the integrated antenna 322, so as to enable the insertable cardiac monitor 300 to communicate with one or more external devices located external to the device 300. For example, as shown in FIG. 5, a cardiac monitoring system 500 may include an insertable cardiac monitor 10 (e.g., insertable cardiac monitor 300), which may include a communication module for communicating with a programmer 510. The programmer 510 may include a user interface that presents information to and receives input from a user. In some variations, the programmer 510 may include, for example, a suitable computing device such as a tablet, a smartphone, desktop computer, laptop computer, and/or the like. It should be noted that the user may also interact with programmer remotely via a networked computing device. As further shown in FIG. 5, in some variations, the insertable cardiac monitor 10 and/or the programmer 510 may be configured to transfer and/or receive information (e.g., cardiac data, such as EGM data and/or cardiac episode-related information derived from the EGM data) to and/or from a secondary memory storage device 520, such as over a wired or wireless network.

A user, such as a physician, technician, surgeon, electrophysiologist, other clinician, or patient, interacts with programmer to communicate with insertable cardiac monitor 300. For example, the user may interact with programmer to retrieve physiological or diagnostic information from the insertable cardiac monitor 300. A user may also interact with programmer to program the insertable cardiac monitor 300, e.g., select values for operational parameters of the insertable cardiac monitor 300. For example, the user may use programmer to retrieve information from the insertable cardiac monitor 300 regarding the rhythm of a patient heart, trends therein over time, or arrhythmic episodes. In some variations, alerts regarding device status (e.g., health state) and/or regarding type(s) of cardiac episode(s) detection may be provided to the patient or a clinician through the programmer 510, though they may be provided in any suitable manner (e.g., personal smartphone, other computing device, pushed through to an electronic medical record, etc.). The insertable cardiac monitor 300 and the programmer may communicate via wireless communication using any techniques known in the art.

In some variations, the insertable cardiac monitor 300 (which is an example of insertable cardiac monitor 10) can be placed subcutaneously in a patient near or over the patient's heart. For example, in some variations the insertable cardiac monitor 300 can be placed in a subcutaneous pocket located over an intercostal space (e.g., over the 4^{th} intercostal space), and positioned at a desirable angle and/or displacement relative to the patient's sternum (e.g., between about 0 and 45 degrees relative to the sternum, about 2 cm from the left edge of the sternum). Once inserted, the insertable cardiac monitor 300 may go through suitable setup and/or calibration processes.

During operation, the insertable cardiac monitor 300 may be configured to detect cardiac episode types based on EGM signal data and save such information to a suitable memory (e.g., memory 406 in the insertable cardiac monitor 300). The exact type of cardiac episodes to be detected for any given patient may depend at least in part on a designated reason for monitoring (RFM) associated with that patient. For example, a first patient may receive an insertable cardiac monitor 300 for the purpose of monitoring primarily for a first cardiac episode type, while a second patient may receive an insertable cardiac monitor 300 for the purpose of monitoring primarily for a second cardiac episode type different than the first cardiac episode type. In some variations, the detection schemes for the first and second patients may differ based at least in part on each patient's designated reason for monitoring, and may affect the manner in which cardiac episodes are predicted (e.g., detected) from EGM data, and/or the manner in which alerts for cardiac episodes are provided. For example, for the first patient, the insertable cardiac monitor 300 may be configured to provide alerts to the patient or their clinician regarding the occurrence of the first cardiac episode type, while deactivating alerts for the second cardiac episode type and other cardiac episode types (and/or reducing the sensitivity in detection of the other cardiac episode types, such as by changing programmable or otherwise predetermined threshold values in an algorithm that classifies cardiac episodes). In contract, for the second patient, the insertable cardiac monitor 300 may be configured to provide alerts to the patient or their clinician regarding the occurrence of the second cardiac episode type, while deactivating alerts for other cardiac episode types (and/or reducing the sensitivity in detection of the other cardiac episode types). Table 1 below summarizes some example cardiac episode types that may be predicted (e.g., detected) from EGM data by the insertable cardiac monitor 300 or other insertable cardiac monitor in accordance with the present technology.

TABLE 1
Example cardiac episode types for detection
by an insertable cardiac monitor

| Cardiac Episode Type | Description |
|---|---|
| Ventricular | The patient's heart rate increases to a rate |
| tachyarrhythmia (VT) | that is higher than a predetermined threshold |
| | for a predetermined tachy duration |
| Fast ventricular | The patient's heart rate increases to a rate |
| tachyarrhythmia (FVT) | that is higher than a predetermined threshold |
| | and/or with an interval lower than another |
| | predetermined threshold, for x of n beats |
| Bradyarrhythmia | The patient's heart rate falls to a rate that |
| (Brady) | is lower than a predetermined threshold for |
| | a predetermined brady duration |
| Asystole (Pause) | No ventricular events are sensed for a |
| | predetermined period of time |
| Atrial | The patient has an atrial fibrillation |
| afibrillation (AF) | determined based on analysis of irregularity |
| | of ventricular rhythm. |
| Atrial | The patient has an atrial tachyarrhythmia |
| tachyarrhythmia (AT) | determined based on analysis of irregularity |
| | of ventricular rhythm. |

Depiction of different features as modules is intended to highlight different functional aspects and does not necessarily imply that such modules must be realized by separate hardware or software components. Rather, functionality associated with one or more modules may be performed by separate hardware, firmware and/or software components, or integrated within common hardware, firmware and/or software components.

Furthermore, it should be understood that the systems and methods described herein in accordance with the present technology are not limited to the cardiac monitor 300 described herein with respect to FIGS. 3 and 4. Rather, the systems and methods described herein in accordance with the present technology may additionally or alternatively be used in conjunction with other cardiac monitor devices (e.g., other leadless cardiac monitoring devices, cardiac monitoring devices with leads, etc.).

### II. Methods of Evaluating Cardiac Monitoring Devices

As described above, a cardiac monitoring device may be evaluated at least in part on the EGM signal provided by the cardiac monitoring device, and in some variations based at least in part on metrics associated with cardiac episodes that are predicted or otherwise determined based on such EGM signal. Example methods for evaluating a cardiac monitoring device are described in further detail below and may be used to characterize a cardiac monitoring device similar to insertable cardiac monitor 300, or any suitable cardiac monitoring device.

In some variations, such methods (e.g., methods 100 and 200) may be performed over a given time period of interest in which the cardiac monitoring device is monitoring the patient. For example, state of a cardiac monitoring device may be evaluated throughout an operational lifetime, beginning at initial activation of the cardiac monitoring device once placed in or on a patient. As another example, state of a cardiac monitoring device may be evaluated over a period of time beginning at any suitable timepoint of interest (e.g., after a cardiac treatment is given to the patient), or in response to a manual or automatic trigger (e.g., a clinician suspicion that the cardiac monitoring device may be faulty), analyzing EGM data for a preceding period of time on a rolling basis (e.g., continuously analyzing the preceding 10 seconds of data). Furthermore, such methods may be performed continuously or intermittently (e.g., periodically). In some example variations, such methods (e.g., methods 100 and/or 200) may be performed continuously beginning at implantation of an insertable cardiac monitoring device in a patient, thereby gathering and analyzing EGM data continuously throughout the lifetime of the cardiac monitoring device.

At least a portion of such methods (e.g., methods 100 and 200) may be performed on-board the cardiac monitoring device (e.g., with processor 404), and/or may be performed by one or more processors located external to the patient, such as part of programmer 510 or other suitable computing device(s).

### A. Health State

FIG. 1 is a schematic flowchart an example method 100 for evaluating a state of a cardiac monitoring device. As shown in FIG. 1, the method 100 may include receiving an EGM signal from a cardiac monitoring device 110, predicting a plurality of potential cardiac episodes experienced by the patient based on the EGM signal 120, and characterizing a health state of the cardiac monitoring device 130, based at least in part on the plurality of predicted potential cardiac episodes.

Receiving an EGM signal from a cardiac monitoring device 110 functions to receive a signal representative of the patient's cardiac electrical activity. The EGM signal may, for example, be generated by electrodes on a cardiac monitoring device inserted in a patient (e.g., inserted in a subcutaneous pocket near or over the heart). The raw EGM signal from the cardiac monitoring device may undergo suitable signal processing (e.g., filtering) for analysis purposes.

The EGM signal may be analyzed for predicting a plurality of potential cardiac episodes experienced by the patient 120. For example, in some variations the EGM signal may be analyzed to detect one or more cardiac episodes, which may be classified as one of the cardiac episode types shown in Table 1. The prediction of cardiac episodes experienced by the patient may utilize one or more suitable algorithms. Such prediction algorithm may utilize, for example, one or more predetermined thresholds for various EGM metrics of interest. In some variations, at least some of the predetermined thresholds and other predetermined values (e.g., referenced in Table 1) used in the prediction algorithms may be programmable (e.g., modified) such as by a clinician or automatically with an adjustment algorithm (e.g., as described with respect to method 200), and/or may be set as default values such as during manufacturing or at initial setup of the cardiac monitoring system. At least some of the predetermined threshold values may be patient-specific, such as individualized to the patient and/or based on a demographic characteristic of the patient (e.g., sex, weight, BMI, cardiac condition, etc.). Additionally or alternatively, the prediction algorithm may include one or more suitable machine learning algorithms (e.g., neural network) that receive EGM data as an input and output the identification and/or classification of one or more cardiac events as a particular cardiac episode type.

In some variations, a VT episode may be detected in EGM data if the patient's heart rate increases to a rate that is higher than a predetermined VT threshold, for a predetermined threshold duration of time associated with VT analysis. In some variations, the predetermined VT threshold and/or the predetermined threshold duration of time associated with VT may be patient specific.

Additionally or alternatively, in some variations a FVT episode may be detected in EGM data if the patient's heart rate increases to a rate that is higher than a predetermined FVT threshold for at least a minimum threshold proportion of recent heartbeats (e.g., x beats of last n beats exhibiting at least the predetermined FVT heart rate threshold). As an illustrative example, a FVT episode may be detected if the patient's heart rate increases to a rate that is higher than about 231 beats per minute (bpm) (intervals lower than 260 ms) for at least 30 of the last 40 beats. However, any suitable predetermined FVT threshold and/or heartbeat proportion threshold may be used to detect FVT, and one or both may be patient specific.

In some variations, a brady event may be detected in EGM data if the patient's heart rate falls to a rate that is lower than a predetermined brady threshold, for at least a predetermined threshold duration of time associated with bradyarrhythmia. In some variations, the predetermined brady threshold and/or predetermined threshold duration of time associated with bradyarrhythmia may be patient specific.

In some variations, asystole may be detected in EGM data if no ventricular events are sensed for a predetermined period of time (e.g., five seconds, ten seconds, etc.). The predetermined period of time for asystole detection may be patient specific.

In some variations, atrial fibrillation and/or atrial tachyarrhythmia may be detected in EGM data based on an analysis of any irregularity of ventricular rhythm, using a suitable algorithm.

Charactering a health state of the cardiac monitoring device 130 functions to evaluate the cardiac monitoring device for any conditions that may indicate a need for corrective action with respect to the cardiac monitoring device. As described in further detail herein, the characterization of the device's health state may, for example, be related to quality of the EGM signal that is provided by the cardiac monitoring device and/or other performance level (e.g., degradation) of the cardiac monitoring device (e.g., caused by hardware and/or software issues in the cardiac monitoring device).

In some variations, the health state of the cardiac monitoring device may be based at least in part on one or more device metrics associated with the plurality of predicted potential cardiac episodes and/or EGM data, as plotted longitudinally over time. Conceptually, in some variations, for a cardiac monitoring device that may have developed problematic issues in quality or device performance, there may be a sudden and sustained change in one or more such device metrics associated with the predicted cardiac episodes. As such, the analysis of device metrics may function to gauge for any sudden and sustained change in the device metrics, which may be considered a “tipping point” for the cardiac monitoring device and indicative of low performance or quality of the cardiac monitoring device. For example, if one or more metrics exhibit a certain amount of change in their daily values all within a certain number of days of each other, the cardiac monitoring device may be considered as potentially in a state of poor health (e.g., low performance or quality).

Generally, in some variations the device metrics of interest when characterizing the state of the cardiac monitoring device may include one or more example device metrics that are summarized in more detail in Table 2. However, other suitable device metrics derived directly or indirectly from cardiac episodes and/or EGM data may be used to characterize state of the cardiac monitoring device.

TABLE 2
Example device metrics for evaluating health state of device

| Device Metric | Description |
|---|---|
| Daily # of FVT | Daily number of potential FVT episodes that |
| episodes | are confirmed as predicted FVT episodes |
| Daily # of rejected | Daily number of potential FVT episodes that, |
| FVT episodes (RejFVT) | upon further analysis, are recharacterized |
| | as noise (e.g., due to very high amplitude |
| | signals and rapidly changing inflection |
| | from positive to negative values) |
| Daily # of VT | Daily number of potential VT episodes that |
| episodes | are confirmed as predicted VT episodes |
| Lifetime # of FVT | Lifetime number of potential FVT episodes |
| episodes | that are confirmed as FVT episodes |
| Lifetime # of rejected | Lifetime number of potential FVT episodes |
| FVT episodes (RejFVT) | that, upon further analysis, are |
| | recharacterized as noise (e.g., due to |
| | very high amplitude signals and rapidly |
| | changing inflection from positive to |
| | negative values) |
| Daily # of Noise | Daily number of intervals during which the |
| Blanking Intervals | measure of apparent “heartbeats” in |
| (NBI) recorded | the EGM signal are rejected as heartbeats |
| | (e.g., due to recorded intervals being too |
| | short to be considered real heartbeats) |
| Duration spent in | Total daily amount of time spanned by FVT |
| FVT episodes | episodes (e.g., in hours) |
| Duration spent in | Total daily amount of time spanned by VT |
| VT episodes | episodes (e.g., in hours) |
| Noise level observed | Noise level observed in the signal for a |
| in ECG/EGM signal | period of interest (e.g., the 10-second |
| | “current” signal sent in a transmission |
| | file from the cardiac monitoring device). |
| | Noise level in the current ECG strip may be |
| | assessed via manual review by clinical data |
| | experts, and/or via an automated algorithm |
| | that assesses noise level, such as signal- |
| | to-noise ratio, high frequency content, |
| | number of peak detections, etc. |

In some variations, the characterization of health state of the cardiac monitoring device may be performed in real-time, or substantially in real-time, with the collection of an EGM signal with a cardiac monitoring device of interest. Additionally or alternatively, the characterizing the health state of the cardiac monitoring device 130 may be performed any suitable time after the EGM signal is collected by the cardiac monitoring device (e.g., as a device diagnostic performed hours or days after the EGM signal is collected by the cardiac monitoring device).

In some variations, multiple device metrics (e.g., device metrics associated with cardiac episodes such as those summarized in Table 2, and/or EGM data itself) may be analyzed to characterize state of the cardiac monitoring device. However, in some variations a single device metric may be sufficient to characterize a cardiac monitoring device as having a potential issue. For example, in some variations characterizing the health state of the cardiac monitoring device may include flagging the cardiac monitoring device as faulty if a detection rate of predicted cardiac episodes (e.g., of a certain type, such as any of the cardiac event summarized in Table 1) satisfies a predetermined threshold (e.g., exceeds or is below the predetermined threshold). The predetermined threshold may, in some variations, be a programmed value, such as a value that is patient-specific or specific to a demographic population to which the patient belongs. In some variations, characterizing the health state of the cardiac monitoring device may include flagging the cardiac monitoring device as faulty if a detection rate of predicted cardiac episodes (e.g., of a certain type) satisfies a condition that is dynamically based on historical trend data for cardiac episode detection for that patient (e.g., daily number of cardiac episodes suddenly jumps or drops by X % over a defined period of time), which may indicate a sudden over-sensing or under-sensing of cardiac episodes that is caused by a device issue.

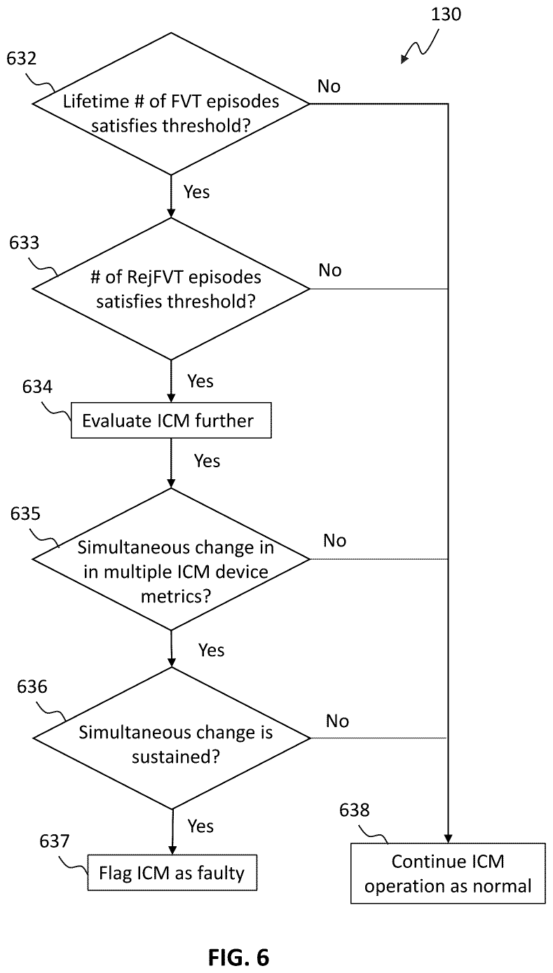

FIG. 6 is an illustrative schematic of an example process for characterizing health state of a cardiac monitoring device 130. In this example, the device state characterization may include determining whether a lifetime number of FVT episodes is above a predetermined threshold (e.g., at least 40 FVT episodes) (block 632) and determining whether a lifetime number of rejected FVT episodes is above a predetermined threshold (e.g., at least 300 rejected FVT episodes) (block 633). These two conditions in block 632 and block 633 may be considered as establishing a “noise floor” that functions as a rough filter to initially gauge whether to further evaluate the cardiac monitoring device (block 634). In some variations, the data used for blocks 632 and 633 may be from a single daily transmission file for a cardiac monitoring device.

The further evaluation of the cardiac monitoring device may, in general, assess whether the device exhibits a pattern of “good” performance across device metrics, then suddenly transitions to “bad” performance across some or all device metrics. In the example of FIG. 6, the analysis may include determining whether there is a simultaneous change in multiple device metrics (block 635), such that occurring within the same day or within a short period of multiple days (e.g., within two days or three days). For example, device metrics of interest for block 635 may include daily # of FVT episodes, daily # of rejected FVT episodes, duration spent in FVT episodes, noise level observed in EGM signal in a daily 10-second “current” signal for the device. However, any suitable device metrics relating to cardiac episodes and/or EGM data (including other metrics summarized in Table 2) may be analyzed for change in block 635.

Where there is a significant simultaneous change in multiple device metrics, the analysis may further include assessing whether that simultaneous change is sustained for a threshold period of time (block 636). For example, in some variations, the simultaneous change in multiple device metrics may be considered as sustained if the change for each device metric during a threshold period of time (e.g., at least two days, at least three days, at least four days, at least five days, at least a week etc.) following onset of the sudden change does not vary more than a certain amount (e.g., no more than 2%, or no more than 5% than the value at onset of the sudden change). In some variations, the data used for blocks 635 and block 636 may be from multiple (e.g., all) daily transmission files for a cardiac monitoring device, which may provide a full longitudinal history of device metrics. If the simultaneous change in multiple device metrics for a cardiac monitoring device is determined to be sufficiently sustained, then the cardiac monitoring device may be flagged as potentially faulty (block 637).

If, however, any one or more of the conditions outlined in blocks 632, 633, 635, and 636 are not met, then the cardiac monitoring device may be considered as performing adequately (block 638), and operation of the device may continue normally.

As described above, the method 100 may further include recommending an action based on the health state of the cardiac monitoring device 140. For example, where a device is flagged as faulty, the recommended action may include removal and/or replacement of the cardiac monitoring device, and optionally return of the cardiac monitoring device to a clinician or to the manufacturer (e.g., in a product recall of related or similar cardiac monitoring devices that have been sold or otherwise distributed) for device investigation. As another example, where a device is flagged as faulty, the recommended action may include further close monitoring of the device (e.g., by a clinician) to gauge appropriate further action relating to the device.

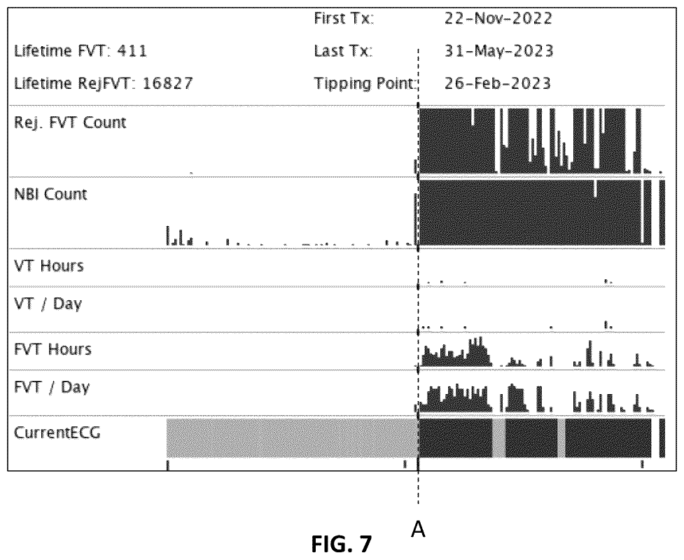

Example implementations of method 100 are described in further detail below with respect to FIGS. 7-11.

### B. Detection Settings

FIG. 2 is a schematic flowchart of another example method 200 for evaluating a state of a cardiac monitoring device. As shown in FIG. 2, the method 200 may include receiving an EGM signal from a cardiac monitoring device 210, predicting a plurality of potential cardiac episodes experienced by the patient based on the EGM signal 220, and recommending a change in one or more settings of the cardiac monitoring device 230, based at least in part on the plurality of predicted potential cardiac episodes. Some processes of method 200 may be similar to method 100. For example, in some variations, receiving an EGM signal 210 may be similar to receiving an EGM signal 110 as described with respect to FIG. 1, and predicting a plurality of potential cardiac episodes experienced by the patient 220 may be similar to predicting a plurality of potential cardiac episodes experienced by the patient 120 as described with respect to FIG. 1.

Recommending a change in one or more settings of the cardiac monitoring device 230 functions to guide modifications to the operation of the cardiac monitoring device, such as based at least in part on a detection rate of cardiac episodes over a period of time. In some variations, analyzing the detection rate of cardiac episodes may additionally or alternatively provide insight into any device performance or quality issues, similar to that described above with respect to method 100. Although the change in settings of the cardiac monitoring device as described primarily as a recommended change (e.g., presented as a suggestion in a dialog box on the programmer, which may be confirmed or accepted through a user input), in some variations the recommended change may be automatically implemented.

In some variations, the recommended change may relate to activation and/or deactivation of alerts that provide notification of detection of a particular type of cardiac episode experienced by the patient. For example, the recommended change may include activating alerts for detection of a particular cardiac episode type, in response to a detection rate of that particular cardiac episode type being below a predetermined threshold for a predetermined period of time. For example, alerts for a cardiac episode type may previously be deactivated or off, based on some assumption, but alerts may be activated since it may be desirable to know when that assumption is incorrect and results in a deviation from an expected baseline. As an illustrative example, a patient having atrial fibrillation (AF) may undergo an AF treatment (e.g., AF ablation), after which it would be expected that few or no AF episodes would be detected (e.g., the detection rate of AF episodes is expected to stop or at least be below a particular predetermined threshold following successful treatment). As such, it may be desirable to be alerted if AF episodes reappear following the AF treatment in order to detect any new AF-related issues with the patient despite that AF treatment. Accordingly, the method may include recommending that alerts for AF episodes be activated (e.g., turned on), in response to the detection rate of AF episodes falling below a predetermined threshold.

As another example, the recommended change may include deactivating alerts for detection of a particular cardiac episode type, in response to a detection rate of that particular cardiac episode type being above a predetermined threshold for a predetermined period of time. For example, if a certain cardiac episode type occurs at a sufficiently high rate for a sustained period of time, it may be reasonable to assume that cardiac episodes of that type will continue to occur, in which case it may be desirable to turn off alerts for that cardiac episode type, so as to reduce distraction, etc.

Additionally or alternatively, the recommended change may relate to changing the specificity of an algorithm for detecting certain cardiac episode types. For example, in response to a detection rate of a certain cardiac episode type exceeding a predetermined threshold, it may be desirable to make the detection algorithm more discriminating as to what kind of EGM data qualifies as a cardiac episode of that type (e.g., increasing or otherwise adjusting one or more threshold values for the EGM data, etc.). Increasing the specificity of the detection algorithm with respect to a particular cardiac episode type that appears to be occurring very frequently may, for example, help filter out “false positives” or inaccurate detection of that cardiac episode type.

In some variations, the recommended change may include allocating more device memory (e.g., in the memory device in the cardiac monitoring device) associated with storing particular kinds of data. For example, in response to a detection rate of a certain cardiac episode type exceeding a predetermined threshold, it may be desirable to earmark more device memory for storing EGM data relating to that cardiac episode type, in anticipation of even more data in the future associated with that cardiac episode type.

In some variations, the recommended change may include modifying a detection profile for detecting cardiac episodes. For example, in some variations, in response to the type of potential cardiac episodes being detected (e.g., new cardiac episode types that were infrequently or never previously detected), the recommended change may include a modification to the reason for monitoring associated with the patient. As an illustrative example, the initial reason for monitoring a patient may be stroke-related following the patient experiencing a stroke; however, if AF events are subsequently and newly detected for that patient, then the reason for monitoring may be modified to be AF-related since AF events may be of greater interest. In some variations, modifying the detection profile may include activating alerts for one or more cardiac episode types (e.g., to focus attention on higher priority cardiac episode types of interest and/or deactivating alerts for one or more cardiac episode types (e.g., to decrease attention on higher priority cardiac episode types of interest, reduce false detections, etc.). Additionally or alternatively, modifying the detection profile may result in changing detection thresholds for detecting certain episode types, with detection of certain episode type being prioritized by different specificity (e.g., thresholds). In this manner, modifying the reason for monitoring in response to newly detected cardiac episode types may help focus the detection scheme on higher priority episode types (and avoid oversaturating a clinician with undesired or irrelevant information).

### III. Examples

FIGS. 7-11 are example plots of device metrics derived from longitudinal EGM data for respective cardiac monitoring devices, for use in evaluating state of the respective cardiac monitoring devices.

For example, FIG. 7 is a plot of device metrics for a cardiac monitoring device, including data from Nov. 22, 2022 to May 31, 2023. The device metrics shown in FIG. 7 include lifetime number of FVT episodes (“Lifetime FVT”), lifetime number of rejected FVT episodes (“Lifetime RejFVT”), daily number of rejected FVT episodes (“Rej. FVT Count”), daily number of Noise Blanking Intervals (“NBI Count”), duration spent in VT (“VT Hours”), daily number of VT episodes (“VT/Day”), duration spend in FVT (“FVT Hours”), daily number of FVT episodes (“FVT/Day”), and a characterization of noise level in the EGM signal (“CurrentECG”). As shown in FIG. 7, there is a sudden, simultaneous and significant change in at least Rej. FVT, NBI Count, FVT Hours, FVT/Day, and CurrentECG, which occurred on or around Timepoint A. Timepoint A is identified as on or around Feb. 26, 2023. Based on the Lifetime FVT, the Lifetime RejFVT, and the change in multiple device metrics around Timepoint A as analyzed in accordance with the techniques described with respect to method 100, the cardiac monitoring device is flagged as faulty, and may warrant further action (e.g., removal from the patient).

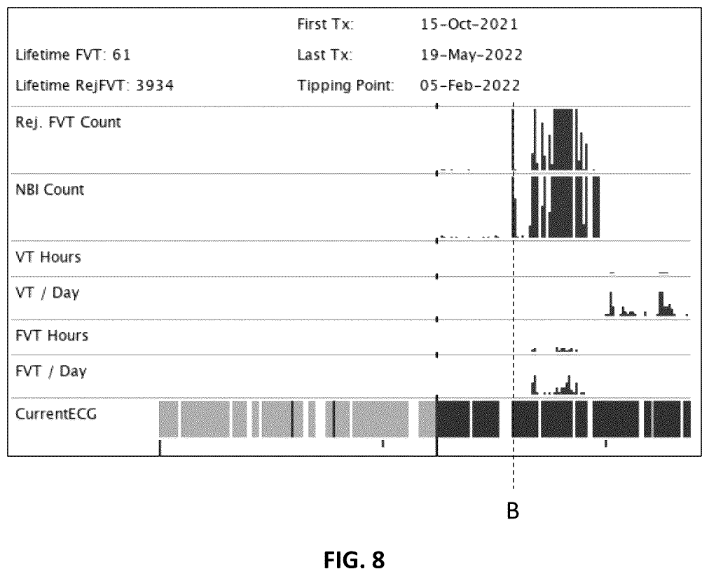

FIG. 8 is another plot of device metrics for a cardiac monitoring device, including data from Oct. 15, 2021 to May 19, 2022. The device metrics shown in FIG. 8 are similar to those shown in FIG. 7 and described above. As shown in FIG. 8, there is a sudden, simultaneous and significant change in at least Rej. FVT Count, FVT/Day, and NBI Count that occurred on or around Timepoint B. Timepoint B is identified as one or around Feb. 5, 2022. There is arguably a temporal delay in the change in such device metrics occurring after the onset of change in CurrentECG. Nevertheless, based on the Lifetime FVT, the Lifetime RejFVT, and the change in multiple device metrics around Timepoint B as analyzed in accordance with the techniques described with respect to method 100, the cardiac monitoring device is flagged as faulty, and may warrant further action (e.g., removal from the patient).

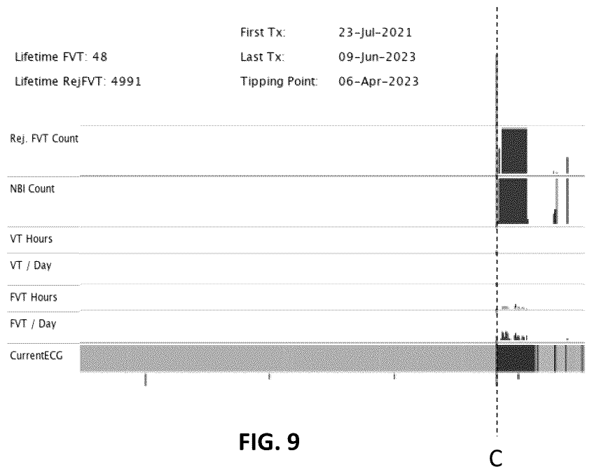

FIG. 9 is another plot of device metrics for a cardiac monitoring device, including data from Jul. 23, 2021 to Jun. 9, 2023. The device metrics shown in FIG. 9 are similar to those shown in FIG. 7 and described above. As shown in FIG. 9, there is a sudden, simultaneous and significant change in at least Rej. FVT Count, FVT Count, NBI Count, and CurrentECG, which occurred on or around Timepoint C. Timepoint C is identified as on or around Apr. 6, 2023. Closer to June 2023, CurrentECG is seen as exhibiting less noise (and reverts to acceptable or negligible noise levels). Nevertheless, based on the Lifetime FVT, the Lifetime RejFVT, and the change in multiple device metrics around Timepoint C as analyzed in accordance with the techniques described with respect to method 100, the cardiac monitoring device is flagged as faulty, and may warrant further action (e.g., removal from the patient).

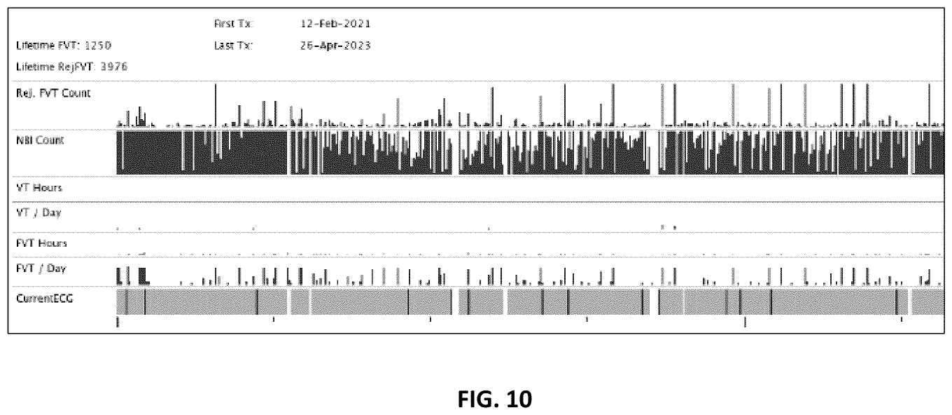

FIG. 10 is a plot of device metrics for a cardiac monitoring device, including data from Feb. 12, 2021 to Apr. 26, 2023. The device metrics shown in FIG. 10 are similar to those shown in FIG. 7 and descried above. While this cardiac monitoring device provided an EGM signal with some amount of noise meeting an initial “noise floor”, the analysis of other device metrics shows that there is no sudden simultaneous transition or change in multiple device metrics, and thus the cardiac monitoring device is not flagged as faulty. More likely, the amount of noise observed in the EGM signal is reflective of intermittent noise that is normal throughout the lifetime of the cardiac monitoring device.

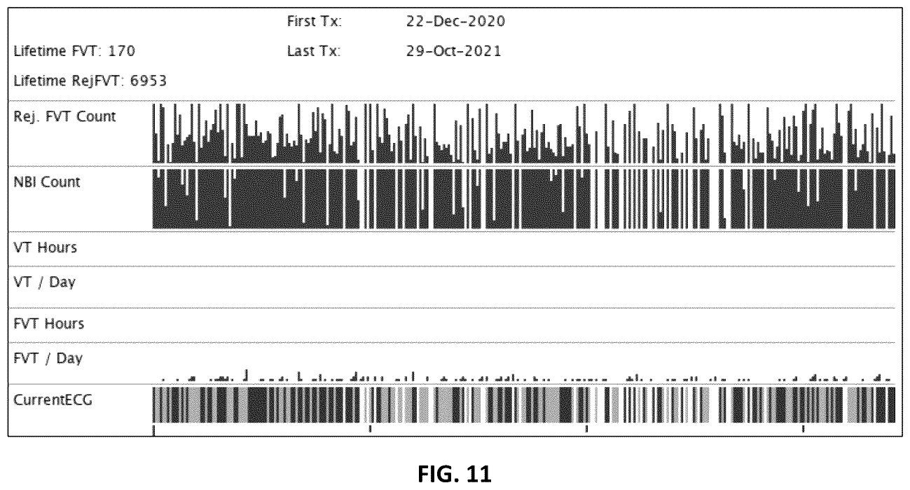

FIG. 11 is a plot of device metrics for a cardiac monitoring device, including data from Dec. 22, 2020 to Oct. 29, 2021. The device metrics shown in FIG. 11 are similar to those shown in FIG. 7 and described above. While this cardiac monitoring device provided an EGM signal with some amount of noise meeting an initial “noise floor”, the analysis of other device metrics shows that there is no sudden simultaneous transition or change in multiple device metrics, and thus the cardiac monitoring device is not flagged as faulty. More likely, the amount of noise observed in the EGM signal is reflective of intermittent noise that is normal throughout the lifetime of the cardiac monitoring device.

### CONCLUSION

Although many of the variations are described above with respect to systems, devices, and methods for evaluating state of a cardiac monitoring device, the technology is applicable to other applications and/or other approaches. Moreover, other variations in addition to those described herein are within the scope of the technology. Additionally, several other variations of the technology can have different configurations, components, or procedures than those described herein. A person of ordinary skill in the art, therefore, will accordingly understand that the technology can have other variations with additional elements, or the technology can have other variations without several of the features shown and described above with reference to FIGS. 1-11.

The descriptions of variations of the technology are not intended to be exhaustive or to limit the technology to the precise form disclosed above. Where the context permits, singular or plural terms may also include the plural or singular term, respectively. Although specific variations of, and examples for, the technology are described above for illustrative purposes, various equivalent modifications are possible within the scope of the technology, as those skilled in the relevant art will recognize. For example, while steps are presented in a given order, alternative variations may perform steps in a different order. The various variations described herein may also be combined to provide further variations.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific variations have been described herein for purposes of illustration, but that various modifications may be made without deviating from the technology. Further, while advantages associated with certain variations of the technology have been described in the context of those variations, other variations may also exhibit such advantages, and not all variations need necessarily exhibit such advantages to fall within the scope of the technology. Accordingly, the disclosure and associated technology can encompass other variations not expressly shown or described herein.

1. A method comprising:
receiving an electrogram (EGM) signal from a cardiac monitoring device inserted in a patient;
predicting a plurality of potential cardiac episodes experienced by the patient, based on the EGM signal; and
characterizing a health state of the cardiac monitoring device, based at least in part on the plurality of predicted potential cardiac episodes.

2. The method of claim 1, wherein characterizing a health state of the cardiac monitoring device comprises at least one of assessing quality of the EGM signal or determining a performance degradation of the cardiac monitoring device.

3. The method of claim 1, wherein characterizing a health state of the cardiac monitoring device comprises evaluating a set of one or more device metrics associated with the plurality of predicted potential cardiac episodes, with a portion of the EGM signal received over a period of interest, or both.

4. The method of claim 3, wherein characterizing a health state of the cardiac monitoring device comprises determining whether the cardiac monitoring device is providing an EGM signal having at least a threshold amount of noise, wherein the threshold amount of noise is associated with at least one of the device metrics.

5. The method of claim 4, wherein the threshold amount of noise is associated with at least one of:
number of potential fast-type ventricular tachyarrhythmia episodes that are confirmed as a fast-type ventricular tachyarrhythmia episode (confirmed FVT episode); or
number of potential fast-type ventricular tachyarrhythmia episodes that are reclassified as a noise event (rejected FVT episode).

6. The method of claim 5, wherein characterizing a health state of the cardiac monitoring device further comprises, in response to determining that the cardiac monitoring device is providing an EGM signal having at least a threshold amount of noise:
evaluating a pattern of the set of device metrics; and
flagging the cardiac monitoring device as faulty if the pattern of the set of device metrics includes a simultaneous change in a plurality of device metrics.

7. The method of claim 6, wherein evaluating a pattern of the set of device metrics comprises evaluating one or more of: number of confirmed FVT episodes per day, number of rejected FVT episodes per day, duration of confirmed FVT episodes per day, or noise level in the portion of the EGM signal received over the period of interest.

8. The method of claim 6, wherein characterizing a health state of the cardiac monitoring device comprises flagging the cardiac monitoring device as faulty if the simultaneous change in the plurality of device metrics is sustained for at least a threshold period of time.

9. The method of any claim 1, wherein characterizing a health state of the cardiac monitoring device comprises flagging the cardiac monitoring device as faulty if an occurrence rate of predicted cardiac episodes exceeds a predetermined threshold.

10. The method of claim 1, further comprising recommending an action based on the health state of the cardiac monitoring device.

11. The method of claim 10, wherein the recommended action comprises at least one of removal of the cardiac monitoring device from the patient, or issuing a recall of distributed cardiac monitoring devices.

12. A system comprising:
a processor;
a memory operably coupled to the processor and storing instructions that, when executed by the processor, cause the system to perform operations comprising:
receiving an electrogram (EGM) signal from a cardiac monitoring device inserted in a patient;
predicting a plurality of potential cardiac episodes experienced by the patient, based on the EGM signal; and
characterizing a health state of the cardiac monitoring device, based at least in part on the plurality of predicted potential cardiac episodes.

13. The system of claim 12, wherein characterizing a health state of the cardiac monitoring device comprises at least one of assessing quality of the EGM signal or determining a performance degradation of the cardiac monitoring device.

14. The system of claim 12, wherein characterizing a health state of the cardiac monitoring device comprises evaluating a set of one or more device metrics associated with the plurality of predicted potential cardiac episodes, a portion of the EGM signal received over a period of interest, or both.

15. The system of claim 14, wherein characterizing a health state of the cardiac monitoring device comprises determining whether the cardiac monitoring device is providing an EGM signal having at least a threshold amount of noise, wherein the threshold amount of noise is associated with a set of one or more device metrics.

16. The system of claim 15, wherein the threshold amount of noise is associated with at least one of:
number of potential fast-type ventricular tachyarrhythmia episodes that are confirmed as a fast-type ventricular tachyarrhythmia episode (confirmed FVT episode); or
number of potential fast-type ventricular tachyarrhythmia episodes that are reclassified as a noise event (rejected FVT episode).

17. The system of claim 15, wherein characterizing a health state of the cardiac monitoring device further comprises, in response to determining that the cardiac monitoring device is providing an EGM signal having at least a threshold amount of noise:
evaluating a pattern of the set of device metrics; and
flagging the cardiac monitoring device as faulty if the pattern of the set of device metrics includes a simultaneous change in a plurality of device metrics.

18. The system of claim 17, wherein characterizing a health state of the cardiac monitoring device comprises flagging the cardiac monitoring device as faulty if the simultaneous change in the plurality of device metrics is sustained for at least a threshold period of time.

19. The system of claim 12, wherein characterizing a health state of the cardiac monitoring device comprises flagging the cardiac monitoring device as faulty if a detection rate of predicted cardiac episodes exceeds a predetermined threshold.

20. The system of claim 12, wherein the cardiac monitoring device comprises a subcutaneous cardiac monitoring device.
